(19) United States
(12) Patent Application Publication
(10) Pub. No.: US 2003/0161805 A1
(43) Pub. Date: Aug. 28, 2003

(54) Organosilicon treated cosmetic powders, their production and use

(75) Inventors: David Schlossman, Short Hills, NJ (US); Yun Shao, Piscataway, NJ (US); Charles A. Quinn, Purdys, NY (US)

Correspondence Address: HANDAL & MOROFSKY, 80 WASHINGTON STREET, NORWALK, CT, 06854, US

(73) Assignee: Kobo Products, Inc., South Plainfield, NJ (US)

(21) Appl. No.: 10/293,745

(22) Filed: Nov. 13, 2002

Related U.S. Application Data
(60) Provisional application No. 60/333,041, filed on Nov. 16, 2001.

(51) Int. Cl. A61K 7/06; A61K 7/11

(52) U.S. Cl. 424/70.12

(57) ABSTRACT

Novel organosilicon-treated cosmetic powders, for example, a pigment, extender pigment or filler are free from residual hydrogen, have a smooth feel, good skin adhesion, good color and spreadability and resistance to acids and alkalis. The treated powders are useful in cosmetics such as powder formulations, oil-in-water and water-in-oil emulsions, anhydrous make-up and lipstick. Treatment can be effected with a linear reactive alkylpolysiloxane having substituted in repeating units in the backbone of the molecule both cationic and anionic groups, for example aminoethylaminopropyl and alkoxy groups. The organosilicon compound can be adsorbed and chemically bonded to the surface of the powder by heat treatment. The alkylpolysiloxane compound can have a degree of polymerization of from 5 to 100, preferably 10-15. A process for producing the treated pigment is also disclosed as are cosmetic formulations made with the treated pigment. The inventive treatment is effective for a wide range of cosmetic powders including inorganic pigments, organic lakes and hard-to-coat powders such as mica-based powders, porous silica and the like.

### CROSS REFERENCE TO RELATED APPLICATIONS

This application claims priority of U.S. provisional patent application No. 60/333,041, filed Nov. 16, 2001.

### STATEMENT REGARDING FEDERALLY SPONSORED RESEARCH OR DEVELOPMENT

(Not applicable.)

### BACKGROUND OF THE INVENTION

The present invention relates to novel organosilicon-treated powders, to a process for the production of organosilicon treated powders, and to coating formulations, especially cosmetic formulations comprising organosilicon treated cosmetic powders. The organosilicon-treated powder particles of the invention are water repellent, or “hydrophobic” and have excellent properties of dispersibility.

Insoluble powder materials, for example colorful pigments, sunscreen agents, talc and the like, are commonly employed in the cosmetics and other industries, such as the paint, coatings and plastics industries, to serve a variety of purposes. Suitable powders may impart qualities of color, opacity or special visual effects, such as pearlescence, or other qualities such as bulk, feel and oil absorbency, to a wide range of consumer and industrial products. Such powders are generally insoluble in either aqueous or organic media. The invention will be described herein as it applies to cosmetic powders, with the understanding that the novel materials, methods and compositions of matter provided by the invention may be useful in other industries where such powders may be employed, as will be apparent to those skilled in the art from this disclosure.

Cosmetic powders of interest to the invention are finely divided particles which are intended to be uniformly dispersed in the finished product. Fine particle size and uniformity of dispersion are desirable characteristics that contribute to the quality of the finished product and to efficient utilization of the powder. Finer powder particles expose more surface area of particle material in the end product, enabling the particles' color or other property to be more efficiently imparted to the finished product. Uniform dispersion of the particles in liquid or even powder excipients is desirable or even essential to provide a consistent commercial product with good shelf life which is free of discoloration, settling or other blemishes.

Some examples of cosmetics product in which the treated powders of the invention may be employed include makeups, lipsticks, blushes, eyeshadow, mascara. Many other products are known to those skilled in the art. The inventive powders may also be employed in other industrial products such as paints and plastics where the particle material is customarily used and where hydrophobic properties are beneficial.

A number of difficulties may arise in uniformly dispersing powders, especially finely divided powders. Untreated, many powders, for example metal oxides such as iron oxide, titanium dioxide and zinc oxide, have significant surface reactivity which may be attributable to chemical reactivity either covalent or ionic, or to more physical phenomena such as adsorbability or accumulation of surface charge. Such surface reactivity may interfere with the uniformity of an initial dispersion of the powder or may adversely impact the long-term stability of the end product. The powder particles may tend to couple covalently or electrochemically with other ingredients in the formulation or to agglomerate, which is to say to stick to each other in agglomerations or clumps. The result may be a poor or unacceptable end product or a product which has limited shelf life owing to non-uniformity of color or other properties, agglomeration, a poor, gritty or sandy feel, settling and so on.

To overcome these problems, it has long been customary to surface treat cosmetic and other powders to render them hydrophobic and to enhance the performance of the powders in finished products. Typical coatings work by reducing the surface activity of the powder particles, repelling water or other aqueous media, inhibiting agglomeration and enhancing dispersibility of the powder particles in aqueous or oily media used in formulating finished products. A satisfactory coating should cover each particle completely and more or less uniformly.

To these ends, many hydrophobic coatings and treatments are commercially available and have been proposed in the literature, especially in the patent literature. Many may be effective for some purposes on one or a small number of cosmetic powders, but no treatment known to applicant is effective on a full range of cosmetic powders.

Insoluble cosmetic powders include many quite different materials such as metal oxides, metal silicates, other inorganic salts, pigment extenders or fillers such as talc and silica as well as organic materials such as lakes, which are organic dyes fixed on metallic salts, and other materials, as is well known in the art. These materials have a variety of surface properties, and a single formulation may use a number of different such powder ingredients, having a number of different coatings. However, the different coatings may interact undesirably with one another. Therefore, to avoid interactions and for simplicity, would be desirable for all the particulates in a given formula to receive the same treatment. It would be still more desirable to have a single hydrophobic treatment which were effective for most regularly used cosmetic powders.

Silicone compounds are noted for their hydrophobicity and have therefore been used as coating materials for cosmetic and other powders. Known hydrophobic treatments for cosmetic powders, notably inorganic and organic pigments and fillers, include a number of organosilicon compounds, for example dimethylpolysiloxanes having a backbone of repeating —Me₂SiO— units (“Me” is methyl, CH₃), methyl hydrogen polysiloxanes having a backbone of repeating —MeHSiO— units and alkoxysilanes of formula RₙOSiH(_{4- n)} where “R” is alkyl and “n” is the integer 1, 2 or 3. The resultant organosilicon-treated pigments or fillers are useful in cosmetic products such as long-lasting liquid makeup and other two-phase, oil-in-water or water-in-oil cosmetics.

To obtain good hydrophobicity, the prior art suggests chemically bonding, or otherwise covering each powder particle with a layer of molecules that will present an external surface consisting essentially of strongly hydrophobic, saturated groups made up entirely of carbon, hydrogen and silicone atoms. Exemplary compounds comprise hydrogen, methyl, ethyl and/or longer alkyl chains coupled to a siloxy backbone of repeating —Si—O— units. Clearly, the presence of other atoms such nitrogen, oxygen or chlorine could bring undue chemical reactivity or water affinity “hydrophilicity” to the coating. The organosilicon molecules can be attached to the powder substrate through the residues of terminal reactive groups provided in suitable starting materials, for example oxygen atoms derived from hydroxy or alkoxy functional groups or hydrolyzed chloro groups.

Witucki in “A silane primer: Chemistry and Applications of Alkoxy Silanes” Journal of Coatings Technology 65;822 pages 57-60 (July 1993) discusses use of alkoxy functional silanes for surface treatment of inter alia particulate pigments and fillers. Described reaction mechanisms include hydrogen bonding to surface hydroxy groups followed by drying or curing with elimination of water to form a covalent bond from each alkoxy-bearing silicon atom to the particle substrate.

In this vein, Hollenberg et al. U.S. Pat. No. 5,143,722 describes the coating of cosmetic pigments with hydrophobic materials comprising dimethylpolysiloxane materials, including cross-linked products. The coatings are prepared from liquid polymerizable silicone starting materials having reactive terminal groups such as hydroxyl or alkoxy groups, by heating slurries of the pigment particles mixed with the starting materials.

Published Japanese patent application JPA 7-196946 (Miyoshi Kasei KK) discloses the use of a straight chain alkylpolysiloxanes having reactive terminal groups such as alkoxy, hydroxy, halogen, amino or imino groups for treating pigments. A similar approach for coating cosmetic powders is disclosed in Hasegawa U.S. Pat. No. 5,458,681, where alkylpolysiloxanes with a specific narrow distribution of molecular weight are employed, namely a ratio of weight-average molecular weight to number average molecular weight of from 1.0 to 1.3.

Use of organometallic to catalyze a surface coating reaction is undesirable, because the presence of materials containing heavy metals in cosmetic products that are applied to the human body is unacceptable, and in many cases illegal.

A number of patents, for example, Hollenberg et al. supra, column 3, lines 43-48, also describe a process of coating pigments with methyl hydrogen polysiloxane and a water-in-oil emulsion comprising such treated pigments. The resulting treated pigment has good water-repellency but suffers the drawback of a tendency to gradually release hydrogen over time.

Methyl hydrogen polysiloxane has Si—H groups which can react with hydroxy groups on the pigment surface. During the coating process, methyl hydrogen polysiloxane may undergo polymerization to form a crosslinked resin coating the particles and possibly also causing cohesive aggregation of the pigments. In this process, the Si—H groups in the methyl hydrogen polysiloxane cannot completely react owing to conformational energy barriers. Residual Si—H groups may then react with the pigment gradually over time, or with other ingredients in the finished product, to release hydrogen, spoiling the integrity of the product.

Another pigment coating process employing methyl hydrogen polysiloxane is described in Horino et al. U.S. Pat. No. 6,200,580. Horino et al., discloses, inter alia use of a reactive alkyl polysiloxane having a single, terminal reactive group, which can be an amino group (column 6, lines 60-65) to coat powdered base materials including sericite. Again, the presence of residual Si—H groups in the end product is undesirable, potentially leading to release of hydrogen gas deleterious to the end product.

A further drawback to the use of methyl hydrogen polysiloxanes, such as Dow Corning (trademark) product #1107, as coating materials for cosmetic powders, is the limited range of materials they can coat. For example, methyl hydrogen polysiloxane does not bond well to lakes of organic colorant such as D&C Red No. 6 Barium Lake and the resulting water repellency is poor. Thus, methyl hydrogen polysiloxanes are unsatisfactory coating materials for cosmetic powders.

Some additional popular organosilicon compounds used as starting materials in the surface treatment of cosmetic powders are alkoxysilanes for example alkyltriethoxy or alkyltrimethoxysilanes such as SILQUEST (trademark) A-137 silane available from OSI Specialities or PROSIL 9202 available from PCR. According to the manufacturer, SILQUEST A-137 silane is a monomeric alkyl alkoxysilane that when exposed to moisture is reactive with the minerals in concrete, masonry and other substrates to penetrate and protectively coat the substrate particles.

Hollenberg et al. supra also teaches, at column 3, lines 32-35 and lines 52-57, an anhydrous pigment coating process employing trialkoxysilanes. Mitchell et al. in U.S. Pat. Nos. 5,486,631; 5,562,897 and 5,756,788 describe anhydrous processes for coating particulate metal oxides such as zinc oxide and titanium dioxide, employing tri(alkoxypolysiloxy)silanes. However, alkoxysilanes are undesirable coating agents for the purposes of the present invention. Specifically, alkoxysilanes hydrolyze when heated in the presence of moisture to yield silanol groups that condense, crosslink and couple to hydroxy groups on the powder substrate surface. Good hydrophobicity can be achieved but crosslinking can cause aggregation of coated pigment particles. Also, if the alkoxysilane is not fully hydrolyzed in the coating process, the residue can react slowly over time adversely affecting the bonds to the pigment surface which can be a problem even, when an anhydrous coating process is employed.

None of the above-described or organosilicon compounds or any other compounds known to applicant is effective with a full range of useful cosmetic powders and few, if any, can effectively coat hard-to-coat materials. For example, none of these compounds can effectively coat highly porous silica, such as the Kobo product, referenced above, to make the porous silica sufficiently hydrophobic to resist water without agglomerating. Porous silica has high oil absorbency and can be used to control oil or as a carrier for an active ingredient such as fragrance.

Another class of materials that are hard to coat is mica-based materials such as sericites which are favored in cosmetics for their pearlescence. Even an alkoxysilane, such as SILQUEST (trademark) A-137 silane from OSI Specialities, Greenwich Conn., one of the more reactive silicone starting materials, does not react well with sericite and the resultant hydrophobicity is not satisfactory.

Glausch et al. U.S. Pat. No. 6,176,918 discloses a method of coating mica-based modified pearl luster pigments employing an oligomeric silane system. In contrast to the objectives of the present invention, Glausch et al.'s coating is intended to provide hydrophilicity, not hydrophobicity. For this purpose Glausch et al. employ an oligomer having silicon-functional hydroxyl groups to bond the silane to hydroxyl groups on the pigment surface. Also, organofunctional groups are provided to bond the silane system to the polymer of the waterborne coating system, i.e. the polymer present in the ink, paint, cosmetic or the like. The organofunctional groups may include amino groups for bonding to polymers such as polyurethane. (See column 5, lines 9-20). The oligomeric silane system comprises no more than four siloxy units, (column 4, lines 59 to column 5, line 8, Formula IX, noting column 5, line 5, a+b+c+d≦4).

Glausch et al.'s process comprises treating pigments which already have one coating of metal oxide with the oligomeric silane system by reaction in aqueous medium (column 4, lines 11-17). The so-modified pigments are then dried (column 4, lines 24-26) and are essentially free of organic solvents (column 4, line 51). The resultant treated pigments are reportedly suitable for water-thinnable coating systems, for example, printing inks, plastics, cosmetics and automotive paint systems (column 7, lines 36-40). Oil dispersibility and water resistance are neither taught nor suggested and are inimical to Glausch et al.'s objective of suitability for water thinning.

Accordingly, there is a need for a hydrophobic treatment process for coating a wide range of cosmetic powder materials which process, preferably can also be used to effectively treat hard-to-coat cosmetic powders. There is a further need for such treated powders in end-product cosmetic and other formulations.

### BRIEF SUMMARY OF THE INVENTION

It is an object of the present invention to solve the problem of providing a versatile coating process that is suitable for coating a wide range of cosmetic and other powders, for example lakes of organic colorant as well as inorganic pigments and fillers, which process provides the powders with desirable hydrophobic properties.

It is another object of the present invention to provide a range of novel, hydrophobically coated cosmetic and other powders which differ as to their substrate particle, inorganic, organic, pigment, lakes of organic colorant, filler, and the like and yet which have generally similar hydrophobic coatings with common, although not necessarily identical, chemical characteristics.

A further object is to provide such a range of coated powders with hydrophobic properties rendering them suitable for incorporation in cosmetic and other end-product formulations, the coated powders being readily dispersible in oils, contributing to good shelf life of the end-product, without inducing gas formation, and providing good end-product esthetic qualities.

A still further object is to provide one or more such coated powders with coatings that are stable to a wide range of pH, for example from pH 4 to pH 9.

It is a still further object of the invention to provide such a product and process which can effectively treat commonly employed hard-to-treat cosmetic powders, for example, sericites and porous silica, which treatments result in products that have excellent hydrophobicity, and which do not release hydrogen.

To solve the aforesaid problem, and to fulfil the above and other objects, the invention provides a process for rendering a powder, optionally a cosmetic powder, hydrophobic, the process comprising treating the powder with an effective amount of an organosilicon coating agent, the organosilicon coating agent comprising a basic organosilicon compound being a dialkyl polysiloxane substituted with alkoxy groups and with a controlled proportion of basic groups. It is strongly preferred that the organosilicon have no silicon-hydrogen bonds whose presence may result in generation of hydrogen in the end product. Preferably also, the alkoxy groups and the basic group are substituted in the same siloxy unit or units.

With advantage, the organosilicon coating agent can comprise in addition to the basic organosilicon compound a nonbasic organosilicon compound which may also be a dialkyl polysiloxane and which preferably also has alkoxy groups substituted in its backbone.

In a particularly preferred embodiment the basic groups comprise amino groups, for example aminoalkylaminoalkyl groups, the dialkyl polysiloxane is a dimethyl polysiloxane and the organosilicon coating agent further comprises a nonbasic organosilicon compound which is similar to the basic organosilicon compound but lacks the basic groups.

Organosilicon-coated powders such as inorganic and organic pigments and fillers, including hard-to-coat materials such as sericites and porous silica, when prepared in accordance with the invention exhibit excellent hydrophobicity providing good water repellency without liberating hydrogen in desirable end-product formulations. Preferred embodiments provide a smooth feel in end-product cosmetic formulations.

Other objects and benefits of the invention will be apparent from the following detailed description.

### DETAILED DESCRIPTION OF THE INVENTION

Some embodiments of the invention, and of making and using the invention, as well as the best mode contemplated of carrying out the invention, are described in detail below. The following more detailed description of the invention is intended to be read in the light of, or in context with, the preceding summary and background descriptions.

Some objects of the invention include, as described above, provision of good or enhanced hydrophobicity in coated cosmetic and other powders, especially for hard-to-coat powders and for a range of different types of powder so that a common coating may be used for all or most of the particulates, be they pigments or fillers, organic or inorganic, that are used in a given cosmetic formulation.

A further object is to provide hydrophobically coated powders that have good water-repellency and which have a smooth feel and good adhesion to the skin in end-product cosmetic formulations.

Broadly stated, the invention provides novel coated cosmetic and other powders that exhibit good hydrophobicity, and to a coating process for coating the powders. The process employs an organosilicon coating agent which preferably comprises first and second organosilicon compounds, although it may comprise only the first organosilicon compound. The first organosilicon compound is itself a polymer having a backbone comprising a limited number of repeating siloxane units of hydrophobic character. This siloxane polymer, or polysiloxane, bears anionic or electronegative reactive groups, in the backbone units or constituting terminal groups, or both. The reactive groups are preferably alkoxy groups but could be other suitable reactive groups such as hydroxyl, ether, keto, carboxyl, ethylene or chloro groups.

The reactive groups bind to the surface of the powder particles to be coated and may also, under the conditions of the coating reaction bind, to a limited extent, to other molecules of the organosilicon compound, polymerizing it. An objective of the coating process is to coat each powder particle evenly and thoroughly with a uniform coating that essentially leaves no exposed areas on the particle surface which could become sites of undesired reactions, for example, with end product excipients. The number, or proportion, of reactive groups, and the reaction conditions, are controlled to avoid undue cross-reactivity which may lead to agglomeration, sticking together of the coated powder particles, or accretion of undesired excess layers of coating material on the particle.

In addition, the first organosilicon compound carries a suitable number of basic groups, for example amino-containing groups, which basic groups are preferably substituted in the repeating units of the backbone. Preferably also, the substitution is in those repeating units that carry anionic reactive groups. In this latter case the organosilicon compound and the relevant units, may be described as “amphoteric”, being a compound or unit having both acidic and basic characteristics.

The second organosilicon compound has similar structural characteristics to the first organosilicon compound but lacks the basic reactive groups, and is preferably also employed in the coating process.

Both the first and the second organosilicon compounds are preferably liquids and the organosilicon coating agent can comprise a blend of the two liquids.

The amino or other basic group in the first organosilicon compound has a good affinity for powders of interest in practicing the invention, and this affinity enhances the adsorption and the spreading of the organosilicon coating agent on the surfaces of substrate powder particles. The alkaline nature of the amino or other basic group enables the group to catalyze the hydrolysis of alkoxy or other anionic reactive groups in either the first or the second organosilicon compound.

The catalyzed reaction proceeds rapidly with the organosilicon compound or compounds curing fast to form a crosslinked elastic and durable film. Rapid curing facilitated by the basic groups may inhibit particle agglomeration and undesired build-up or accretion of the coating on the powder particles. Rapid curing may also enhance coating hardness, a further desirable characteristic of the coated particles of the invention.

The first, basic organosilicon compound, can, if desired, be used alone and will cure fast and effectively to a hard film. However, employment of the second, non-basic compound, in addition to the first, is preferred for rheological reasons, to enhance the spreading of the organosilicon agent on the powder particles and the effectiveness with which each particle is covered.

The basic groups can be any suitable basic groups that will not interfere with the hydrophobicity of the coated powder. A preferred basic group is an amine functional group. The basic groups can be the same or different in each molecule of the silicone compound and preferably comprise nitrogen-containing alkyl groups or a heterogenous nitrogen-carbon chain. The basic groups can comprise a primary amino group which can terminate an alkyl chain which can optionally also have one or more secondary amino groups in the chain in addition to the terminal primary amino group. Alternatively, the basic group can comprise a secondary or tertiary amine having lower alkyl substituents. A quaternary ammonium group, if employed as a basic substituent in the basic organosilicon compound is preferably present to a relatively low degree in view of the strongly cationic character of quaternary ammonium groups. The organosilicon is preferably also fully saturated and is terminated with alkoxy or alkyl groups.

Preferably the alkyl groups employed in the organosilicon compound are lower alkyl groups having no more than ten carbon atoms. More preferably, referring to nonbasic substituents in the organosilicon compound, the alkyl groups have no more than five carbon atoms and still more preferably are methyl or ethyl groups.

Preferably also, the organosilicon compound is free of reactive groups other than those specified herein. For example, it is preferred that the organosilicon compound be free of hydroxyl, thio, carboxyl, chloro, nitro groups and unsaturation. Thus it is preferred that the organosilicon compound consist essentially of dialkyl siloxy groups, alkoxy groups and basic groups. While it is preferred that the alkyl substituents in the siloxy groups each be the same as the other, most preferably methyl groups or possibly ethyl or other groups, it will be understood that different alkyl groups, for example methyl, ethyl and butyl groups, may be present in the same molecule.

It is contemplated that the substitutions of alkoxy and basic groups in the dialkyl polysiloxane will be made directly into the silicon atoms of the backbone, but it is to be understood that substitution into one or both of the dialkyl groups may be possible, provided that the resultant organosilicon compound meets with the guidelines and objectives of the invention as described herein.

The proportion of siloxy groups without basic groups to siloxy groups bearing basic groups in the organosilicon compound can vary widely, for example from about 5:1 to about 1:5, but is preferably from about 2:1 to about 1:2, more preferably about 1:1.

The number of siloxy groups in the organosilicon compound can vary widely, for example from about 2 to about 200, but is preferably from about 5 to about 100, more preferably from about 5 to about 30 and still more preferably from about 10 to about 15.

Treatment of the cosmetic powders may be effected with a single homogenous basic organosilicon compound as described hereinabove, or with a heterogenous mixture of two or more such basic organosilicon compounds having different structures in accordance selected in accordance with the teachings of the present invention.

A preferred class of basic organosilicon compounds for use in the coating process of the invention comprises compounds complying with the following Formula 1:

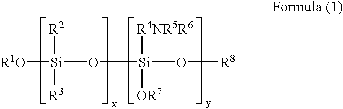

wherein

R¹, R⁷ and R⁸ are independently hydrogen or lower alkyl and are preferably methyl or ethyl;

R² and R³ are lower alkyl and are preferably methyl or ethyl;

R⁴ is a divalent lower alkyl group having formula —CₙH₂ₙ— where “n” is an integer from 1 to 10 preferably from 2 to 4 and more preferably is propylene;

R⁵ is hydrogen or lower alkyl and is preferably hydrogen;

R6 is hydrogen, lower alkyl or amino lower alkyl;

(x+y) is from 5 to 100, preferably from 10 to 15; and

x:y is from about 5:1 to about 1:5, preferably from about 2:1 to about 1:2 and is more preferably about 1:1, optionally about 1.2:1 to 1:1.2.

“Lower alkyl” is used herein to reference an alkyl group having from one to ten carbon atoms, preferably from 1 to 5 carbon atoms and more preferably methyl or ethyl. The value of (x+y) indicates the degree of polymerization and number of units in the polysiloxane.

Particularly preferred Formula 1 compounds are compounds wherein R¹, R², R³, R⁷ and R⁸ are methyl and R⁵ is hydrogen.

A preferred coating composition includes, as organosilicon coating agent, a blend of organosilicon compounds of the following formulas (2) and (3):

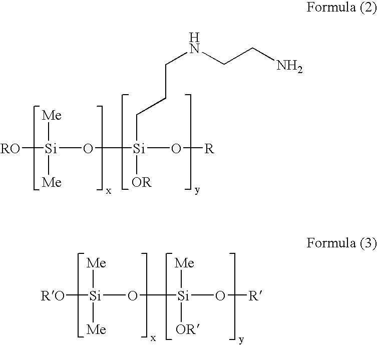

wherein

Me is methyl;

R is methyl or ethyl;

R′ is methyl or ethyl; and

(x+y) is from 5 to 100, preferably from 10 to 15; and

x:y is from about 2:1 to about 1:2 and is more preferably about 1:1, optionally about 1.2:1 to 1:1.2.

The ratio of the compound of formula (2) to the compound of formula (3) in the blend can be any effective proportion, but is preferably in the range of from about 0.2:1 to about 5:1, and preferably about 1:1, for example from about 1.2:1 to about 1:1.2, on a weight basis. One preferred such blend is an amine functional silicone fluid available from GE Silicones, Waterford, N.Y., under the product code SF 1706. According to its data sheet, the product has a viscosity at 25° C. of 10-50 centistokes, a specific gravity at 25° C. of 0.986 and closed cup flash point of 95° C. and an amine equivalent of 0.48 milliequivalents of base/gram. The product has a 100 percent silicone content and is soluble in most aromatic hydrocarbons.

A blend of compounds according to formulas(2) and (3), for example the GE Silicones SF 1706 product, is a particularly effective organosilicon coating agent for hydrophobizing a variety of cosmetic powders. For example, a coating employing such a blended organosilicon coating agent can reduce the surface activity of titanium dioxide, zinc oxide and iron oxide, can facilitates the dispersion of particulate in oil, ester and silicone and can control the color shift that typically occurs with colored pigments when the pigments are wetted.

While preferred embodiments of the organosilicon coating agent have been described as comprising a blend of a basic and a nonbasic organosilicon compound, whose molecular structures have been graphically illustrated, it will be understood that the structures depicted may be indicative but somewhat idealized models and that the actual structures of the compounds employed may vary or comprise a range of variation from the depicted structures, the extent of variation of which will depend upon the method of manufacture of the materials. Also, the blended materials may both be produced in a single process, as is known in the art, rather than being separately produced and then blended.

Suitable Powder Materials. Powder materials suitable for use in the practice of the present invention include a wide range of inorganic pigments and organic pigments, pigment extenders and fillers and especially most if not all insoluble powder materials employed in the cosmetics arts. Preferably, the powder materials employed have a mean particle size of from about 0.01 to about 100 μm, preferably from about 0.01-20 μm. Powder materials having a mean particle size of from about 0.1 to about 10 μm are particularly useful.

Some suitable inorganic pigments for use in the practice of the present invention include titanium dioxide, zinc oxide, iron oxide, alumina oxide, chromium oxide, manganese violet, ultramarines and metal oxide composites of one metal oxide with another metal oxide or with an inorganic salt.

Other inorganic pigments may be employed, as known to those skilled in the art, for example:

white pigments including lithopone, zinc sulfide, zirconium oxide, barium metaborate, Pattinson white, manganese white, tungsten white, magnesium oxide, and the like;

black pigments including carbon black, titanium black, silica black, graphite, and the like; gray pigments including zinc dust, zinc carbide, and the like;

red pigments including cobalt red, molybdenum red, cobalt magnesia red, cuprous oxide, copper ferrocyanide, and the like;

yellow pigments including ocher, iron oxide yellow, titanium yellow, barium yellow, strontium yellow, chrome titanium yellow, aureolin (cobalt yellow), tungsten yellow, vanadium yellow, nickel yellow, and the like;

green pigments including chrome green, chromic oxide, chromic hydroxide, zinc green, cobalt green, cobalt-chrome green, Egyptian green, manganese green, Bremen green, titanium green, and the like;

blue pigments including ultramarine, Prussian blue, cobalt blue, tungsten blue, molybdenum blue, Egyptian blue, Bremen blue, copper borate, lime blue and the like; and

violet pigments including Mars violet, manganese violet, cobalt violet, cobalt violet, chromic chloride, copper violet, ultramarine violet, and the like.

brown pigments including umber, brown iron oxide powder, Vandyke brown, Prussian brown, manganese brown, copper brown, cobalt brown and the like; and

metal powder pigments including aluminum powder, copper powder, bronze powder, stainless steel powder, nickel powder, silver powder, gold powder and the like.

Some suitable organic pigments for use in the practice of the present invention include aluminum, barium, calcium and zirconium lakes of FD&C and D&C grades of Red No. 6, Red No. 7, Red 21, Red No. 27 and Yellow No. 5.

Other suitable organic pigments may be employed, as known to those skilled in the art, for example pigments incorporating various aromatic dyes such as azo, indigoid, triphenylmethane, anthraquinone, hydroquinones and xanthine dyes, and other D&C and FD&C colors as well as the lakes of these colors, as are known in the art.

Suitable pearlescent pigments include titanated mica, fish scale white, bismuth oxychloride, titanated mica treated with iron oxide, mica titanium treated with Prussian blue, titanated mica treated with carbon black, titanated mica treated with carmine, and the like.

Suitable fillers include talc, mica, sericite, kaolin, barium sulfate, calcium carbonate, silica, hydroxyapatite and polymeric powders. The silica can be porous or non-porous silica in various shapes, including spherical, ellipsoidal, rod-like, irregular and other shapes as known to those skilled in the art. Suitable polymeric powders include polymethyl methacrylate, cellulose and nylon powder which may optionally be microporous and/or coupled to other particles to form a shell-like complex, for example as described and claimed in Schlossman U.S. Pat. Nos. 5,356,617 and 5,314,683.

Other suitable fillers or pigment extenders include silica white, barium carbonate, magnesium carbonate, magnesium silicate, calcium silicate, barium sulfate precipitated, baryte, alumina white, gypsum, clay, satin white, bentonite, magnesia, slaked lime, strontium white and the like.

The invention is of particular value when multiple different powders coated pursuant to the invention are employed in a single formulation, for example a cosmetic formulation. In this way problems of incompatibility between different coatings are avoided. The multiple different powders may comprise any desired combination of powders required by the formulation, for example one or more inorganic pigments together with one or more organic pigments. The formulation may also, or alternatively, include one or more each of a pearlescent pigment or a pigment extender, or both. Of particular interest are multiple powders comprising an inorganic or organic pigment, and a hard-to-coat pigment such as a mica-based pigment, e.g. a sericite, or a porous silica, or both, or both an inorganic pigment, an organic pigment and a hard-to-coat pigment together optionally with a pearlescent pigment if not included in one of the foregoing categories. The particular inorganic, organic or hard-to-coat pigment or pigment extender or filler can be one of the products described herein or other such products, as known to or discovered by those skilled in the art.

Proportion of Coating Agent to Powder. The proportion of organosilicon coating agent used to treat the powder to be coated in practicing the present invention will depend upon the nature of the substrate and should be sufficient to provide desired properties such as water repellency, smooth feel and good adhesion to the skin but not so much as to make the pigment too wet or to tend to cause agglomeration. A suitable proportion, based upon the weight of the coated pigment, filler or other powder or particulate material to be coated, is from about 0.1 to about 30 percent, preferably from about 1 to about 10 percent and more preferably from about 2 to about 5 percent.

Coating Process. Any suitable process may be used for coating the cosmetic powders with the organosilicon coating agent. However, a preferred hydrophobizing process comprises the following process elements:

a) thoroughly mixing the organosilicon coating agent with the particulate powder material to be coated, preferably in a liquid dispersion medium;

b) filtering the resulting slurry to remove excess liquid and yield a paste;

c) heating the paste to remove residual liquid components, cure the coating and yield a dry coated powder material; and

d) pulverizing the dried powder to the desired particle size.

Mixing element a) can be effected in various ways, as will be understood by those skilled in the art. For example, employing an aqueous dispersion medium, a liquid organosilicon coating agent can be added to an aqueous slurry of the powder to be coated in the dispersion medium.

Alternatively, and preferably, the organosilicon coating agent is dissolved in a suitable organic solvent for example isopar, especially isopar C and the solution is sprayed onto the powder and mixed well. Isopar is a partially neutralized mixture of isoparaffinic acids and isopar C comprises C7-C8 solvents. Other suitable solvents for the organosilicon coating agent may be employed as known to those skilled in the art, for example different grades of isopar, such as isopar E or isopar G, isoheptane, isooctane, isononane, and petroleum distillates such as those available from Phillips Chemical under the trade names or trademarks Soltrol 130, Soltrol 150 and Soltrol 170.

As is well understood in the art, mixing should be continued until the mixture is well mixed, smooth and uniform.

Heating of the paste is effected at any suitable temperature, preferably at a temperature of between about 60 and about 130° C., under vacuum for from about two to about ten hours until dry, as may be determined by weight loss determination, if desired.

Pulverization of the dried powder is effected in conventional manner for example using a mill, such as a jet mill, hammer mill, or the like.

Coated powders. Coated powders according to the invention preferably comprise a thin, coherent homogenous film of organosilicon coating agent covalently bound to the particulate substrate. The coated particles may be made by a coating process as described herein, or by other processes known to those skilled in the art, or that subsequently become known to those skilled in the art, and that are suitable for use with the materials described herein. The coating is hydrophobic and preferably completely covers each particle, preventing ingress of reactive chemical agents, aqueous media, wetting agents, excipients or other ambient materials in the environment of the coated powder to the substrate particle material beneath the coating.

While the invention is to be limited not by any particular theory but only by the claims appended hereto, the molecular structure of the coated particles may be understood to comprise a web of cross-linked organosilicon agent residues, many most or preferably all of which residues are also covalently bound to the substrate. The bonds between neighboring residues and the substrate are largely, or entirely effected through oxygen atoms derived from one or more of the alkoxy groups R¹O—, R⁷O—, R⁸O—, RO— or R′O— in the organosilicon agent molecule. The resultant links between adjacent residues may be Si—O—Si links and the links between the residues and the particle substrate are Si—O—P groups where P is an atom in the substrate having an available valence, for example, in the case of an inorganic powder, a metal. Alternatively, in the case of an organic or organic-laden powder, such as a lake, P may be a carbon atom. As a further, though less probable or less frequent, alternative, the connecting moiety between the silicon atom and the metal or carbon atom may be a peroxy —O—O— group, the additional oxygen atom being derived from an available OH— group in surface moisture on the powder or from an organic hydroxyl group.

Referring for example to the compounds shown in formulas (2) and (3), with the understanding that other organosilicon agent compounds may participate in an equivalent manner, the film structure may include combinations of: two or more terminally linked organosilicon agent residues; two or more organosilicon agent residues linked backbone-to-backbone, through respective repeating unit alkoxy group oxygen atoms; and two or more organosilicon agent residues linked from the terminus of one residue to the backbone of another. These composite residues may be bonded to the powder substrate through one or more unused alkoxy group oxygen atoms.

The basic or amino group in the organosilicon agent starting material may also provide a link to an adjacent organosilicon agent or the powder substrate, for example an —N—C— or possibly an —NO—C— link. However, it is contemplated that the basic or amino group will in many cases be unreacted, or possibly, hydrated. It is also contemplated that the basic or amino group may serve as a localized buffer or facilitate buffering, enhancing the pH stability of the coated powder product.

The invention can provide powders of excellent stability for cosmetic purposes. Preferred embodiments of inventive coated powder are able to tolerate a variety of cosmetic formulants with good shelf life under customary extremes of ambient temperature and humidity. In particular, they may be resistant to relative extremes of acidity or alkalinity. For example they may be resistant to a pH as low as 4 or even as low as 2 or to a pH as high as 9 or even as high as 10, or resistant to both such low and high pH levels. Such tolerance of pH extremes is of particular value in certain cosmetic products. For example, skin care products containing alpha hydroxy acids may be quite acid and some other products, for example mascara may be significantly alkaline.

Their unique properties render the organosilicon coated powders of the present invention suitable for incorporation in a wide range of cosmetic formulations in proportions known to those skilled in the art, for example, depending upon the product, the cosmetic powder may comprise from 0.1 to 99 percent by weight of the end-product formulation, with lower proportions of from about 0.1 to 25 weight percent being preferred in liquids and creams, more preferably from about 1 to about 10 percent by weight.

The excellent hydrophobicity of the organosilicon coated powders of the invention render them particularly suitable for oil-in-water or water-in-oil emulsions such as creams and lotions, wherein the hydrophobically coated pigments have a strong affinity for the oil phase and do not tend to migrate undesirably to the aqueous phase.

There is no particular limit to the cosmetic product into which the coated powders of the invention may be formulated. Such products include skin care compositions skin packs, sunscreens, body lotions, body powder compositions, makeup, compositions including face powder, foundation, eye shadow, blush, lipstick, eye liner and eye brow and so on.

More than one organosilicon coated powder according to the present invention can be employed in a given cosmetic formulation. Where multiple such powders are employed they may be coated with the organosilicon agent either separately or together. The adaptability of the invention to provide a diverse range of coated powders makes it possible for two, three, four or more different powders, to be coated simultaneously in the same process, in a very efficient manner. Thus for example one or more inorganic pigments, one or more organic pigments, one or more pearlescent or other hard-to-coat pigments and one or more fillers or a combination including two or more of each of the foregoing powder types may be coated simultaneously by premixing the powders together prior to exposure to the organosilicon agent. Because the powder particles all have the same coating, the coatings will not interact in the end product.

Some non-limiting examples, pursuant to the invention, of the preparation of organosilicon coated powders will now be described and compared with prior art treatments.

### EXAMPLE 1

### Porous Silica

95 g of powdered porous silica from Kobo Products Inc. are added to a blender. 25 g of a 20% wt/wt solution of an amine functional silicone fluid (GE Silicones SF1706) in isopar are sprayed on the porous silica powder with agitation. The mixture is thoroughly blended, transferred to a tray and dried at 110° C. for 4 hours. It is then cooled to room temperature and pulverized. The obtained powder shows excellent hydrophobicity.

### COMPARATIVE EXAMPLE A

The procedure of Example 1 is followed employing a similar quantity of methyl hydrogen polysiloxane (Dow Corning DC 1107) in place of the amine functional silicone fluid. The obtained powder has a poor hydrophobicity and cannot float on water after mild shaking, indicating the particle surfaces are wetted.

### EXAMPLE 2

### Sericite

95 g of sericite (GMS 4C manufactured by Kinsei Matec Co. Ltd.) are added to a blender. 13.3 g of a 30% wt/wt solution of amine functional silicone fluid (GE Silicones SF1706) in solution in isopar are sprayed on the powder under agitation. The mixture is thoroughly blended, transferred to a tray and dried at 110° C. for 4 hours. It is then cooled to room temperature and pulverized. The obtained powder shows excellent hydrophobicity. After mixing and shaking with water, the treated powder floats well and the water soon becomes clear.

### COMPARATIVE EXAMPLE B

The procedure of Example 2 is followed employing a similar quantity of methyl hydrogen polysiloxane (Dow Corning DC 1107) in place of the amine functional silicone fluid. The obtained powder has a poor hydrophobicity and cannot float on water after mild shaking, indicating the particle surfaces are wetted.

### EXAMPLE 3

### Titanium Dioxide

98 g of titanium dioxide 328 from Whittaker, Clark & Daniels, Inc. are added to a blender. 6.67 g of a 30% wt/wt solution of amine functional silicone fluid (GE Silicones SF1706) in isopar are sprayed on the powder under agitation. The mixture is thoroughly blended, transferred to a tray and dried at 110° C. for 4 hours. It is then cooled to room temperature and pulverized. The obtained powder shows excellent hydrophobicity. After mixing and shaking with water, the treated powder floats well and the water soon becomes clear.

### EXAMPLE 4

### Barium Lake

95 g of K 7096 D&C Red 6 Barium Lake powder from Les Colorants Wackherr are added to a blender. 16.67 g of a 30% wt/wt solution of an amine functional silicone fluid (GE Silicones SF1706)) solution are sprayed on the pigment powder under agitation. The mixture is thoroughly blended, transferred to a tray and dried at 110° C. for 4 hours. It is then cooled to room temperature and pulverized. The obtained powder shows excellent hydrophobicity. After mixing and shaking with water, the treated powder floats well and the water soon become clear.

Some examples of cosmetics end-product formulations employing pigments coated in accordance with the invention, in which ingredient percentages are by weight based on the weight of the composition, are described below.

### PH Stability Test

1 g samples of each of the coated powder products of Examples 1-4 and Comparative Examples A-B are separately shaken with two 50 g aqueous aliquots. One aqueous aliquot comprises a solution of water with sufficient acid to adjust the pH to 2 and the other aliquot comprises a solution of water with sufficient base to adjust the pH to 10. The ability of the coated powder to float on the aqueous aliquot is indicative of the quality of the hydrophobic coating. The products of Comparative Examples A-B each show some settling or sinking of particles within half an hour and exhibit substantial sinking after two days. In contrast the inventive products of Examples 1-4 exhibit little if any sinking after two weeks, demonstrating excellent stability to both acid and alkaline pH values.

### EXAMPLE 5

### Oil-in-Water Liquid Makeup

EXAMPLE 5
Oil-in-water Liquid Makeup

| | % |
|---|---|
| Part A | |
| Lanolin Alcohol and Mineral Oil | 11.50 |
| Cetyl Esters | 3.20 |
| Stearic Acid | 3.50 |
| Glyceryl Monostearate | 1.80 |
| Talc | 2.00 |
| Titanium dioxide | 4.00 |
| Yellow iron oxide | 1.00 |
| Red iron oxide | 0.40 |
| Black iron oxide | 0.15 |
| Part B | |
| Propylene glycol | 12.00 |
| Triethanolamine | 1.00 |
| PE 20 Sorbitan Monolaurate | 0.65 |
| Magnesium Aluminum Silicate | 1.00 |
| Carboxymethyl Cellulose | 0.30 |
| Deionized Water | 57.20 |
| Preservatives and Fragrance | QS |

The titanium dioxide and iron oxides are surface coated with an organosilicon agent as described in Example 1, until the color is fully developed. The ingredients of Part A are combined, one at a time, in the sequence listed above while thoroughly mixing each component until the mixture is homogenous before adding the next ingredient. The complete mixture of Part A ingredients is heated to 60° C. In a separate vessel, the ingredients of Part B are combined. Part B is slowly added to Part A while mixing well. The product is poured into suitable containers.

### EXAMPLE 6

### Liquid Compact Foundation (Hot Pour)

EXAMPLE 6
Liquid compact foundation (Hot pour)

| | % |
|---|---|
| Part A | |
| Titanium dioxide | 26.76 |
| Red iron oxide | 0.54 |
| Yellow iron oxide | 0.54 |
| Black iron oxide | 0.16 |
| Mica | 10.00 |
| Silica (spherical) | 2.00 |
| Part B | |
| Squalane | 10.00 |
| Dimethicone (5 cst) | 17.00 |
| Octyl hydroxystearate | 7.00 |
| Polyglyceryl-3 diisostearate | 3.00 |
| Microcrystalline wax | 7.00 |
| Octyl palmitate | 7.00 |
| Carnauba wax | 1.00 |
| Part C | |
| Nylon -12 | 8.00 |

The titanium dioxide and iron oxides are surface coated with an organosilicon agent as described in Example 1, until the color is fully developed. The Part A ingredients are then micronized, which is to say pulverized and/or ground to a suitable fine particle size for example between 400 and 800 U.S. mesh. The Part B ingredients are heated, with stirring to about 90-93° C. Stirring is continued for about one half hour. Part A is added to Part B, mixed until homogeneous and cooled to about 82° C. Part C is added and the complete mixture is mixed until homogeneous and poured into pans at about 74-77° C.

### EXAMPLE 7

### Lipstick

EXAMPLE 7
Lipstick

| Ingredient | % |
|---|---|
| Candelilla Wax | 6.00 |
| Carnauba Wax | 3.00 |
| Ozokerite | 4.00 |
| Paraffin Wax | 2.00 |
| Yellow Beeswax | 6.00 |
| Lanolin Alcohol | 6.00 |
| Oleyl Alcohol, | 10.00 |
| BHA | 0.20 |
| Castor Oil | 43.25 |
| D&C Red No. 6 Barium Lake | 2.50 |
| D&C Red No. 7 Calcium Lake | 2.50 |
| Iron Oxides | 1.00 |
| FD&C Blue No. 1 | 0.80 |
| Perfume | 0.75 |
| Pearlescent pigment (titanium dioxide and | 10.00 |
| mica) | |

The barium and calcium lakes and iron oxides are all separately surface coated with an organosilicon agent as described in Example 1, until the color is fully developed. Castor oil is placed in the main mixer and heated to 80° C. using a steam pan. The treated pigments and the dyes are slowly mixed into the castor oil using a Lightnin'mixer under high speed for 30-60 minutes. The candelilla wax, carnauba wax, beeswax, ozokerite, paraffin wax oleyl alcohol and lanolin alcohol are all preheated and melted together at 80-85° C. using a steam pan. The molten wax mixture is added to the castor oil, pigment and dye mixture. Mixing is continued throughout the addition of these ingredients.

The perfume is added with further mixing until the mixture is homogeneous. The pearlescent pigment comprising titanium dioxide and mica previously treated with a titanium coupling agent, for example isopropyl titanium triisostearate (Kobo Products Inc., S. Plainfield, N.J.) is then added and mixing continues until the product is uniform. The lipstick is then cooled and shaped as is customary.

The liquid makeup, foundation and lipstick produced by the processes described in Examples 5, 6 and 7 respectively have a uniform appearance without settling, streaks or discolorations, a smooth feel and good skin adhesion. Because the pigment coatings lack silicon-hydrogen bonds, hydrogen gas generation on the shelf is not an issue.

As may be understood from the foregoing disclosure, the present invention provides a novel cosmetic powder treatment process and novel hydrophobically treated cosmetic powders. Preferred embodiments of the invention can be employed to produce an effective hydrophobic coating on a wide variety of useful and commercially significant cosmetic powders. Excellent or superior water repellency, stability with good shelf life and no outgassing, smooth feel and good adhesion to the skin are obtainable in cosmetic formulations in which preferred embodiments of the invention are employed.

### INDUSTRIAL APPLICABILITY

While the present invention has been particularly described as it applies to novel hydrophobic cosmetics powders and to cosmetic formulations employing such cosmetic powders, it will be understood by those skilled in the relevant art or arts that the invention may be beneficially applied in other industries, for example in the paint and coatings industries and the plastics industry, where powders analogous to cosmetic powders are employed.

While illustrative embodiments of the invention have been described above, it is, of course, understood that various modifications will be apparent to those of ordinary skill in the art. Many such modifications are contemplated as being within the spirit and scope of the invention.

1. A process for rendering a powder, optionally a cosmetic powder, hydrophobic, the process comprising treating the powder with an effective amount of an organosilicon compound having a dialkyl polysiloxane backbone substituted with alkoxy groups and with a controlled proportion of basic groups.

2. A process according to claim 1 wherein the powder is insoluble in aqueous and organic media and the organosilicon agent has no silicon-hydrogen bonds.

3. A process according to claim 2 wherein the organosilicon agent comprises a repeating siloxy unit substituted with one of said alkoxy groups and with the basic group.

4. A process according to claim 2 wherein the organosilicon coating agent comprises a nonbasic organosilicon compound, optionally being a dialkyl polysiloxane having alkoxy groups substituted in its backbone.

5. A process according to claim 2 wherein the basic groups comprise amino groups, optionally aminoalkylaminoalkyl groups, the dialkyl polysiloxane is a dimethyl polysiloxane and the organosilicon coating agent further comprises a nonbasic organosilicon compound the nonbasic organosilicon compound being similar to the basic organosilicon compound but lacking the basic groups.

6. A process according to claim 2 wherein the organosilicon agent is selected from the group consisting of: a compound complying with Formula (1) herein; a compound complying with Formula (2) herein; a compound complying with Formula (1) herein together with a similar compound lacking the basic group; a compound complying with Formula (2) herein together with a similar compound lacking the basic group; and a compound complying with Formula (2) together with a compound complying with Formula (3) herein.

7. A process according to claim 2 wherein the powder comprises one or more powders selected from the group consisting of inorganic pigments, organic pigments, pearlescent pigments, mica-based pigments, hard-to-coat pigments, pigment extenders and fillers.

8. A process according to claim 7 wherein the powder comprises at least one inorganic pigment, at least one organic pigment and a hard-to-coat pigment, the hard-to-coat pigment optionally being selected from the group consisting of sericites, pearlescents, porous silica and mica-based pigments.

9. A process according to claim 8 comprising the following process elements:
a) thoroughly mixing the organosilicon coating agent with the particulate powder material to be coated, preferably in a liquid dispersion medium;
b) filtering the resulting slurry to remove excess liquid and yield a paste;
c) heating the paste to remove residual liquid components, cure the coating and yield a dry coated powder material; and
d) pulverizing the dried powder to the desired particle size.

10. Coated powder produced by the method of claim 1.

11. A coated powder comprising insoluble powder particles coated with a film comprising a cross-linked web of dialkyl siloxy groups and of silicon trioxy groups each bearing a basic group, the film component groups being covalently bonded one to another by oxygen atoms and the film being bonded to the powder also by oxygen atoms.

12. A coated powder according to claim 11 resistant to wetting by an acidic aqueous solution having a pH of about 4 and by an alkaline aqueous solution having a pH of about 9.

13. A method of preparing a cosmetic composition comprising adding an effective amount of a coated powder according claim 11.
